SEQUENCE LISTING (7 sequences)

SEQ ID NO: 1 - protein, 1368 aa, Streptococcus pyogenes strain SF370 [sequence omitted]

SEQ ID NO: 2 - DNA, 4104 bp, Streptococcus pyogenes strain SF370
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 3 - protein, 1227 aa, Acidaminococcus sp BV3L6
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 4 - DNA, 3681 bp, Acidaminococcus sp BV3L6
tcaaagctcgagaaattcaccaactgttattcgttgagcaaaacactgcggtttaaagcgattccagtcggcaagactcaagagaatatagacaataagcggctgttggtggaagatgaaaagcgcgcggaagactacaaaggggtgaagaagttgttggacagatactacctctcttttatcaatgatgtcttgcactcaatcaaattgaagaatctgaacaactacatctccctcttcagaaagaaaacaaggacagaaaaggagaataaggaacttgaaaatttggagatcaatctgaggaaagagatcgcgaaagcctttaaaggcaacgaaggatacaaaagtctgttcaagaaggatataattgagacaattttgccagagttcctcgatgacaaggacgagattgcgctggtcaattcgttcaacggattcacaacagcattcacaggcttctttgataatcgggaaaatatgttctctgaggaggcaaagtccacttctattgcgttcaggtgtatcaatgagaatctcactaggtacatttccaacatggatatctttgagaaggttgacgcaatttttgacaagcacgaagttcaggagattaaggagaagatcctcaattccgattatgacgttgaggacttcttcgaaggtgagttttttaatttcgtgctcactcaagagggtatcgacgtgtataatgcgatcatcggtgggttcgtgactgagtccggtgaaaagattaagggattgaacgagtatatcaacctttacaaccaaaagacgaaacagaagctgccaaagttcaagcctctttacaaacaggttctttcagaccgcgagtcactctcgttctatggggagggctacacttcggatgaggaagtcctggaggtgttcaggaatactctcaataagaattcggagattttctcttctataaaaaaactggaaaagttgtttaagaattttgacgaatactctagcgccggcatatttgtgaaaaacggcccggccatatcaacgataagtaaagatatcttcggcgaatggaacgtgatcagagacaaatggaacgcggagtatgacgatattcacctgaagaagaaggctgtcgtaacggagaagtacgaggatgatcgcaggaaaagcttcaaaaagatcggaagtttcagcctggaacagttgcaggagtatgctgacgccgatcttagcgtcgtcgagaagttgaaggagataatcatccaaaaggtcgacgagatatataaagtctatggatcaagtgaaaaactgttcgacgccgacttcgttttggagaagtccctgaagaagaacgacgctgttgttgccattatgaaggatctgctcgacagcgtgaagagtttcgagaactatattaaggcttttttcggggaggggaaggagactaacagagatgagtccttctacggagacttcgtcctcgcgtacgatatactccttaaggtagaccacatctacgacgcaatcagaaattacgtgacacaaaagccgtacagcaaggacaagttcaaactctacttccagaacccccagttcatgggcggctgggacaaggacaaggaaacggattacagggctacgatcctgaggtatggttcaaaatactacttggcgattatggacaagaagtacgccaagtgtctccagaagattgacaaagacgatgtcaatggcaattatgagaagatcaactacaagctgcttccgggtccgaacaagatgctcccaaaggttttcttcagcaagaaatggatggcctactataacccaagcgaggacatccagaagatttataagaacggtacgttcaagaagggcgacatgttcaatcttaacgactgtcacaagctgatcgacttcttcaaagactcaattagccggtacccaaagtggtctaacgcctatgacttcaacttttcggaaaccgagaagtacaaggatatagccggattttatagagaggtggaagagcagggctacaaggtgtcattcgagtccgccagcaagaaggaagtggacaagctcgtggaagagggtaagctctacatgttccagatttataataaagactttagcgataagagccacgggacacctaatctccacacaatgtatttcaagctgctcttcgacgagaataaccacggccaaatcaggttgtcaggaggggctgaactcttcatgcggcgcgctagccttaagaaggaggagcttgtagtccaccctgcgaatagtccaattgcgaataagaacccggacaatcctaaaaagactacaacattgagctacgacgtgtacaaggataagaggttttccgaggatcagtacgagctccacatcccgattgcgatcaacaagtgcccaaagaatattttcaagataaacacagaggtgcgtgtactcctgaagcatgacgacaatccttacgtcattgggattgatcggggcgagaggaacctcctctatattgtggtggtggacgggaaggggaacatagtcgaacagtactcccttaacgaaataattaacaatttcaacggcatccgtatcaagaccgactaccattcgttgctggacaagaaggagaaggagagatttgaggcgcggcaaaattggacaagtatcgagaacatcaaggaactcaaagcaggttatatctctcaagttgtgcataagatatgcgagctggttgagaagtatgacgcagtgatcgctcttgaggacctcaactcgggctttaagaattctagagttaaagtggagaagcaggtctatcaaaagttcgagaagatgcttatagataagctcaactacatggtcgataagaaatcgaacccatgtgccaccggcggcgcactcaaaggttaccaaataacaaacaaattcgagtccttcaaatcgatgagtactcagaatgggttcatattttatataccggcgtggcttacgtctaagatcgacccgtcaactggttttgtcaacctgttgaagacgaaatacacgtccattgccgattcgaaaaagttcatatctagttttgatcgtattatgtacgtcccagaggaagatcttttcgagtttgctctcgactacaaaaacttttcgcggaccgatgcggattacattaaaaaatggaaactctattcgtacggcaacagaatcaggatttttcgcaaccctaagaagaataacgtctttgattgggaggaagtttgcttgactagcgcgtacaaggagctctttaataagtatggcattaactaccaacagggtgatatcagagcactgctttgcgaacaatctgacaaggctttctactcatccttcatggctttgatgagcctgatgctccagatgagaaattcaattacaggcagaaccgacgtggatttcttgatctccccggttaaaaattctgatggcatcttttacgatagcaggaactatgaagcgcaagagaatgcgattctgccaaaaaatgcagacgccaacggtgcctataacatcgccaggaaagtcctgtgggcgatcggccagttcaaaaaggccgaagacgaaaaattggacaaggtcaaaatcgctatcagcaacaaagagtggctggagtatgctcagacatccgtaaagcat

SEQ ID NO: 5 - DNA, 600 bp, Artificial Sequence
  template DNA used to verify activities of mutant SpCas9s
tccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagccacggcgtccagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacat

SEQ ID NO: 6 - protein, 1638 aa, Artificial Sequence
  AsCas12-BE
MEASPASGPRHLMDPHIFTSNFNNGIGRHKTYLCYEVERLDNGTSVKMDQHRGFLHQQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNHGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSPKKKRKVLEHHHHHH

SEQ ID NO: 7 - DNA, 4917 bp, Artificial Sequence
  AsCas12-BE
atggaagcaagtccagcctcgggccctcgtcacttaatggatccgcatatcttcacgtccaacttcaacaatggcatcggccgtcacaagacctatctttgctatgaagtcgaacgtctggataacggcacgagcgtaaagatggatcaacaccgtggtttccttcaccaacaggctaagaacttattatgtgggttctacggtcgtcacgcagagttgcgcttccttgacctggtaccttcccttcaactggatcctgcacaaatctaccgcgttacctggttcatttcttggagtccatgcttttcttggggctgtgctggcgaagtacgtgcatttttgcaagagaatacgcatgttcgtttgcgtatcttcgcggcccgcatttatgattatgacccgctttataaggaggctttacagatgttacgtgacgctggcgcacaggtttcaattatgacctacgacgagttcaaacactgctgggacacattcgtcgatcatcaaggttgtccctttcaaccctgggatgggttagacgaacacagccaggcgttgtcgggacgcctgcgtgcgattcttcaaaatcaaggtaacagcgggtccgagacacccggaacatcggaaagtgccactcctgagtctatgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatcgcaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcccgtggctgggacgtgaatcgcgagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgcgcggggcgagagaaacctgatctatatcacagtgatcgactccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgagaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaaccatggatcacccaagaagaaacggaaagtgtctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtcctcgagcaccaccaccaccaccactga

(19) United States
(12) Patent Application Publication
(10) Pub. No.: US 2024/0174996 A1
(43) Pub. Date: May 30, 2024

(54) METHOD FOR SITE-SPECIFIC CONJUGATION OF NUCLEIC ACID TO CRISPR FAMILY PROTEIN, AND CONJUGATE THEREOF AND USE THEREOF

(71) Applicant: BEIJING HUIDA BIOTECHNOLOGY CO., LTD, Beijing (CN)

(72) Inventors: Tao LIU, Beijing (CN); Xinyu LING, Beijing (CN); Xiaoqin GAO, Beijing (CN)

(73) Assignee: BEIJING HUIDA BIOTECHNOLOGY CO., LTD, Beijing (CN)

(21) Appl. No.: 17/775,482

(22) PCT Filed: Nov. 11, 2020
(86) PCT No.: PCT/CN2020/127992

§ 371 Date: May 9, 2022

(30) Foreign Application Priority Data
Nov. 11, 2019 (CN) 201911092554.8

(51) Int. Cl. C12N 9/22; C12N 15/113

(52) U.S. Cl. CPC C12N 9/22; C12N 15/113; C12N 2310/20; C12N 2310/3513

(57) ABSTRACT

A method for site-specific conjugation of nucleic acid to a CRISPR family protein, and a conjugate thereof and the use thereof. The site-specific conjugation method comprises: mutating a CRISPR family protein site-specifically with an unnatural amino acid, and then conjugating a nucleic acid to the mutated CRISPR family protein, wherein the unnatural amino acid has orthogonal chemical reactivity. The CRISPR family protein site-specifically mutated and the conjugate thereof of the present application can be used to improve gene cutting, gene editing efficiency and base editing efficiency, etc.

### CROSS-REFERENCE TO RELATED APPLICATION

This application is the U.S. national phase of PCT Application No. PCT/CN2020/127992 filed on Nov. 11, 2020, which claims priority to Chinese Patent Application No. 201911092554.8 filed on Nov. 11, 2019, which are incorporated herein by reference in their entireties.

### SEQUENCE LISTING

The text file AFD_ST25 of size 54,902 bytes created Apr. 17, 2023, filed herewith, is hereby incorporated by reference.

### TECHNICAL FIELD

The application belongs to, but is not limited to, the field of biomedicine, and particularly relates to a method for site-specific conjugation of a nucleic acid to a CRISPR family protein, a conjugate and use thereof.

### BACKGROUND

CRISPR is an RNA-dependent acquired immune defense mechanism in bacteria and archaea, which can effectively help bacteria resist invasion of external DNAs such as phages, etc., cut exogenous genes efficiently and accurately at specific sites, and prevent exogenous genes from expanding in vivo. Bacteria use this mechanism to prevent viral infection, so as to protect themselves. SpCas9 and AsCas12a are derived from Streptococcus pyogenes and Acidaminococcus BV3L6, respectively, and are functional operators of the CRISPR system. By base pairing with a target DNA, crRNA directs Cas9 or Cas12a to the vicinity of a target region, and changes their conformations to become a ribonucleoprotein complex (RNP) with DNA cleavage activity. Activated Cas9 or Cas12a can cleave the target DNA to form a double strand break (DSB). DNA double strand breaks are highly lethal to a cell, and the cell will thus quickly initiate corresponding repair mechanisms, primarily including two repair pathways: non-homologous end joining (NHEJ) and homologous directed repair (HDR). The former is widely used by cells in DNA damage repair due to its no requirement of repair template and the fast response, in which deletions or insertions are introduced as a result; HDR requires a repair template, and this repair pathway can only be initiated when the cell is at a certain stage in division cycle, so as to complete the precise repair of DNA double strand breaks. Exploiting DNA double strand breaks generated by the CRISPR system and under the guide of a template DNA, scientists complete proper editing of a gene. Since the discovery of this system, it has been widely used in genetic modification in animal husbandry and agriculture because of its high efficiency, simplicity and stable editing efficiency, effectively improving the yield and quality of livestock and cash crops. In terms of medical and health, it not only brings hope for the cure of diseases, especially genetic diseases in human, but also promotes the development of basic scientific research, basic medicine and clinical therapeutics.

With the development and advance of biopharmaceutical technologies, random modification to protein has been gradually eliminated due to uniformity in batch production, poor quality controllability and the like reasons. Protein drugs with site-specific modifications have become the research and development hotspot and development direction of biotechnology pharmaceuticals nowadays because of their uniform and controllable properties. At present, common protein site-specific modification technologies mainly include the following categories: 1. the technology using transpeptidase and intein, which depends on enzymatic reaction and can achieve site-specific modification of specific sequences on protein; 2. glycosyltransferase-based site-specific labeling technology, which depends on glycosylation modification and achieves site-specific labeling of protein by fusing a small protein or tag such as Aldehyde Tag and the like to the protein; 3. protease-dependent biotin labeling of specific sequences. However, all types of these labeling require specific sequences, and such specific sequences could only be added at N/C terminus of the target protein, which is prone to affect the stability and function thereof for enzymes and many proteins, leaving a narrow space for selection, and it is not suitable for the modification of Cas9 and Cas12a.

CRISPR technology has currently been successfully applied to genetic engineering in animal husbandry and agriculture and to biomedicine, e.g., treatment of genetic diseases. However, there are still some problems in this technology. For example, with respect to the low efficiency of homologous recombination by SpCas9 in gene editing, it has been reported that increasing the concentration of donor DNA at the double strand break will facilitate improvement in efficiency of gene editing (Ma, M., Zhuang, F., Hu, X., Wang, B., Wen, X. Z., Ji, J. F., and Xi, J. J. (2017). Efficient generation of mice carrying homozygous double-floxp alleles using the Cas9-Avidin/Biotin-donor DNA system. Cell research 27, 578-581). Therefore, scientists make use of the affinity between a fusion protein and its ligand to closely connect donor DNA to Cas9 proteins, so that after cleavage occurs, donor DNA can quickly reach the DSB to guide the completion of homologous recombination repair (Carlson-Stevermer, J., Abdelen, A. A., Kohlenberg, L., Goedland, M., Molugu, K., Lou, M., and Saha, K. (2017). Assembly of CRISPR ribonucleoproteins with biotinylated oligonucleotides via an RNA aptamer for precise gene editing. Nature communications 8, 1711; Hemphill, J., Borchardt, E. K., Brown, K., Asokan, A., and Deiters, A. (2015). Optical Control of CRISPR/Cas9 Gene Editing. Journal of the American Chemical Society 137, 5642-5645; Savic, N., Ringnalda, F. C., Lindsay, H., Berk, C., Bargsten, K., Li, Y., Neri, D., Robinson, M. D., Ciaudo, C., Hall, J., et al. (2018). Covalent linkage of the DNA repair template to the CRISPR-Cas9 nuclease enhances homology-directed repair. eLife 7). While these schemes do improve the efficiency of homologous recombination repair, the fusion of a protein to Cas9 protein will increase the difficulty in transfection and affect the RNP complex that has been effectively delivered into cells. Labeling chemical modification on a relatively long piece of donor DNA also significantly increases synthesis cost and greatly limits its wide application. The above scientific research cannot effectively and extensively solve this fundamental problem. For another example, AsCas12a has also attracted extensive attention in the field of gene editing, since it has a different PAM recognition region from that of Cas9, and that the sticky ends produced after cleavage are more conducive to homologous recombination. However, after being delivered to a cell, AsCas12a has extremely low cleavage efficiency. It is assumed that AsCas12a has a low affinity to its crRNA, and dissociates therefrom during the delivery process, with a very small amount of the RNP effectively delivered to DNA at the target region, thereby causing AsCas12a to have low cleavage efficiency. In addition, the single-base gene editing technology has attracted extensive attention because it can achieve single-site base repair of gene without double strand breaks to be generated in a cell. However, at present, there are still some problems in base editing which limit its application in biomedical field, such as large editing site window, inability to achieve accurate editing, and low editing efficiency.

While CRISPR technology has currently achieved good application results in various fields, there are still some problems caused by e.g., insufficient efficiency of homologous recombination and efficiency of cleavage as mentioned above. Therefore, there is an urgent need to provide a solution to these problems in the art.

### SUMMARY

The following is an overview of the subject matter described in detail herein. This summary is not intended to limit the protection scope of the claims.

In view of the above problems with an urgent need to be solved, the inventors have developed a method for site-specifically conjugating a nucleic acid to a CRISPR family protein via chemical covalent bond, and deliver the protein and nucleic acid as a whole into a cell to exert the gene editing function thereof. In the present application, after the CRISPR family protein is covalently conjugated to donor DNA, the CRISPR protein reaches the position of target DNA for cleavage under the guide of gRNA, and the donor DNA conjugated to the protein may complete correct repair under the action of homologous recombination repair protease after cleavage. Again, in terms of the problem with low in vivo cleavage efficiency of Cas12a, by improving affinity of Cas12a proteins to crRNA, effective RNP complex concentration in the vicinity of the target DNA is improved, which improves cleavage efficiency and may also improve efficiency of base editing, thus solving the problem of low efficiency of accurate gene editing in the prior art.

The inventors exploit the protein translation system of TyrRS (MjPolyRS)/tRNA^{CUA}, PylRS or LeuRS from multi-specific Methanococcus to site-specifically incorporate an unnatural amino acid into a protein, thus obtaining a site-specifically mutated CRISPR protein. In addition, to the CRISPR family protein, an unnatural amino acid containing a bioorthogonal reactive group such as an azide, an alkynyl, an aldehyde, a ketone, a cyclopropyletetrazine or the like structure is introduced at specific sites by gene codon expansion technologies, and a corresponding modified nucleic acid is conjugated by click chemical reaction, thereby solving some problems present in the application of CRISPR family proteins in gene editing.

The present application provides a method for site-specific conjugation of a nucleic acid to a CRISPR family protein, comprising: firstly, mutating the CRISPR family protein site-specifically with an unnatural amino acid, and subsequently conjugating the nucleic acid to the CRISPR family protein; wherein the unnatural amino acid has orthogonal chemical reactivity.

In another aspect, the present application also provides a CRISPR family protein site-specifically mutated by an unnatural amino acid, wherein the unnatural amino acid has orthogonal chemical reactivity.

In a third aspect, the present application also provides a site-specific nucleic acid conjugate of a CRISPR family protein, wherein the CRISPR family protein is a CRISPR family protein site-specifically mutated by an unnatural amino acid, and the unnatural amino acid has orthogonal chemical reactivity.

In a fourth aspect, the present application also provides a method for preparing the CRISPR family protein site-specifically mutated by an unnatural amino acid described above, a site-specific nucleic acid conjugate thereof, and a nucleic acid sequence corresponding to the CRISPR family protein site-specifically mutated by an unnatural amino acid, a vector and a host expression cell thereof.

In a fifth aspect, the present application provides use of the CRISPR family protein site-specifically mutated by an unnatural amino acid described above, or the site-specific nucleic acid conjugate thereof.

### BRIEF DESCRIPTION OF DRAWINGS

The accompanying drawings are used to provide a further understanding of the technical schemes of the present application, and constitute a part of the specification. They are used to explain the technical solutions of the present application together with the embodiments of the present application, and do not constitute a limitation to the technical schemes of the present application.

FIG. 1: expression of SpCas9-G1367 having unnatural amino acid AeF and purification by nickel column affinity chromatography.

FIG. 2: SpCas9-G1367-AeF protein was further purified by cation exchange following purification by nickel column. The arrow in the left panel indicates the SpCas9-G1367-AeF protein eluted under the condition of gradient salt concentration; and the right panel shows SDS-PAGE identification for protein fractions collected at the arrow.

FIG. 3: SpCas9-G1367-AeF was further purified by molecular sieving following the ion exchange. The arrow in the left panel indicates SpCas9-G1367-AeF protein in monomer; and the right panel shows SDS-PAGE identification for protein fractions collected at the arrow, and the protein has a purity over 95%.

FIG. 4: evaluation of in vitro cleavage efficiency of SpCas9-G1367-AeF. Control represents uncut template DNA, SpCas9-WT is wild-type protein and gRNA, and G1367-AeF is SpCas9-G1367-AeF protein and gRNA. The black lines indicate two pieces of DNAs formed by RNP after cleavage. As can be seen from the results, the in vitro activity of SpCas9-G1367-AeF protein has no substantial difference from that of the wild-type.

FIG. 5: exploration of the reaction conditions for SpCas9-G1367-AeF protein and DBCO modified DNA. Reaction times for lanes 1-4 are 0 h, 1 h, 2 h and 3 h, respectively. As can be seen, click chemical reaction is substantially 100% completed at 3 h.

FIG. 6: evaluation of in vitro activity of SpCas9-G1367-AeF and SpCas9-K1151-AeF in conjugation of oligo DNA.

FIG. 7: evaluation of in vitro activity of AsCas12a-M806-AeF in conjugation of crRNA.

FIG. 8: evaluation of in vitro activity of SpCas9-G1367-AeF in conjugation of oligo DNA, in which: control represents untreated group, G1367-AeF represents SpCas9-G1367-AeF protein and gRNA being transfected into cell, and G1367-AeF-NH₂-oligo represents NH₂ modified oligo DNA and donor DNA being added after SpCas9-G1367-AeF protein and gRNA forming the RNP. G1367-AeF-DBCO-oligo represents DBCO modified oligo DNA and donor DNA being added after SpCas9-G1367-AeF protein and gRNA forming the RNP.

FIG. 9: evaluation of in vivo activity of AsCas12a-M806-AeF in conjugation of crRNA.

FIG. 10: effect of covalent conjugation of oligo DNA on efficiency of gene cleavage of RNP after incubation with donor DNA.

FIG. 11: effect of covalent conjugation of crRNA to RNP of Cas12a on precise gene repair realized by precise knock-in of a short fragment. As can be seen from the results, complex in the conjugation group is capable of significantly improving the editing efficiency.

FIG. 12: construction of a base editing system containing dAsCas12a, and as shown in panel A, targeted editing area is marked in red, and panel B shows that covalent conjugation of crRNA to RNP of Cas12a can effectively improve the efficiency of site-specific base editing.

### DETAILED DESCRIPTION

In an embodiment of the present application, the present application provides a method for site-specific conjugation of a nucleic acid to a CRISPR family protein. The method includes:
(a) mutating the CRISPR family protein site-specifically with an unnatural amino acid;
(b) conjugating the nucleic acid to the CRISPR family protein site-specifically mutated by the unnatural amino acid obtained in step (a), thereby obtaining a site-specific nucleic acid conjugate of the CRISPR family protein;
wherein the unnatural amino acid has orthogonal chemical reactivity. Here, orthogonal chemical reactivity means, without limitation, that the unnatural amino acid contains a group such as an azide, an alkynyl, an aldehyde, a ketone, a tetrazine or a cyclopropene group.

In an embodiment of the present application, the CRISPR family protein includes, but is not limited to, Cas9 protein, Cas12a protein, CasX protein, Casφ protein, Cas12g protein from different species or genera, or a related inactivated counterpart thereof having no cleavage activity but retaining binding activity (such as dCas9 protein, dCas12a protein, or ddCas12a protein, etc.). Here, the Cas9 protein may be SpCas9 derived from Streptococcus pyogenes strain SF370, SauCas9 derived from Staphylococcus aureus, NmeCas9 derived from Neisseria meningitidis, or St1Cas9 derived from S. thermophilus 1. The Cas12a protein may be selected from FnCas12a derived from Francisella novicida U112, LbCas12a derived from Lachnospiraceae bacillus ND2006, AsCas12a derived from Acidaminococcus sp. BV3L6, or MbCas12a derived from Moraxella bovoculi 237. The CasX protein may be DpbCasX derived from Deltaproteobacteria CasX, or PlmCasX derived from Planctomycetes CasX. Preferably, the CRISPR family protein is Cas9 protein or Cas12a protein, preferably SpCas9 or AsCas12a. In the present application, Cas12a and Cpf1 are used interchangeably.

In some embodiments of the present application, the unnatural amino acid may be selected from one of the following compounds:

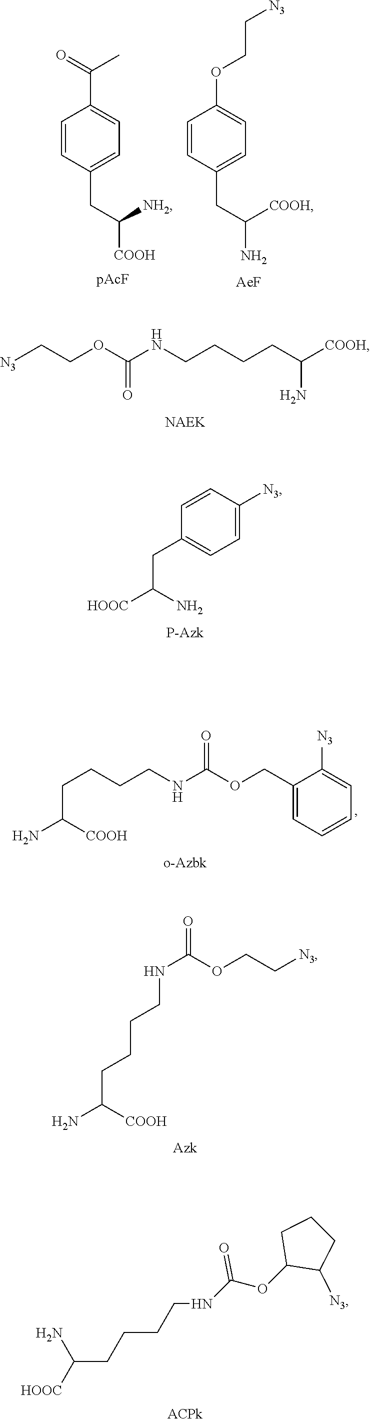

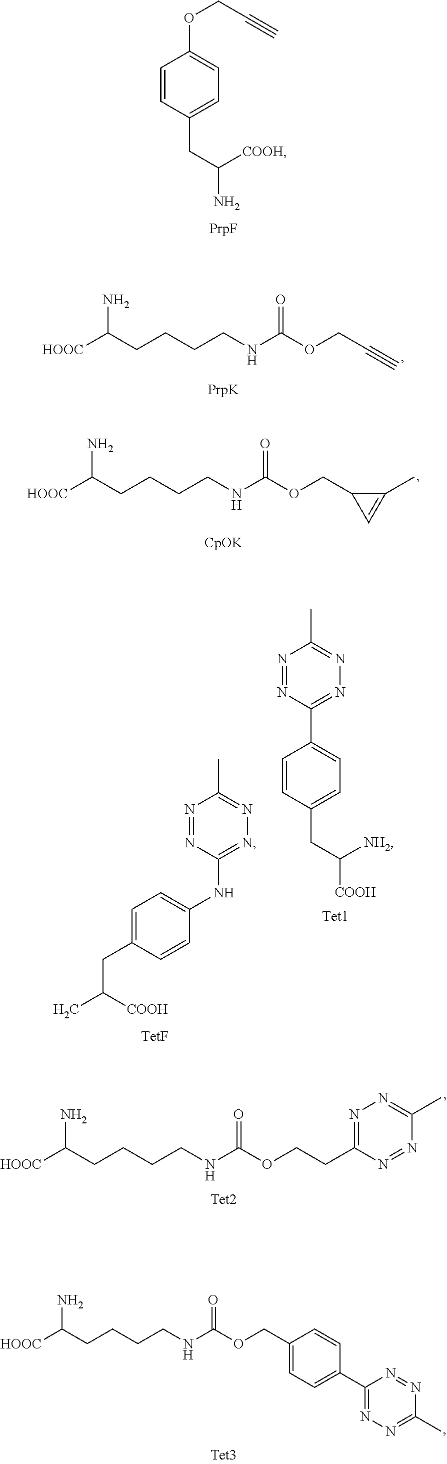

preferably pAcF, AeF, PrpF, NAEK or Tetf.

In some embodiments of the present application, the present application also provides a CRISPR family protein site-specifically mutated by an unnatural amino acid; wherein the unnatural amino acid bears a group such as an azide, an alkynyl, an aldehyde, a ketone, a cyclopropene or a tetrazine group;

Optionally, the mutation on SpCas9 is at one or more of the following sites: positions K3, D39, H41, H116, D576, E945, K1151 and G1367 of SEQ ID NO. 1;
the mutation on AsCas12a is at one or more of the following sites: positions M806, L834, S835, K860, K1086 of SEQ ID NO. 3.

In an example of the present application, the present application provides a CRISPR family protein site-specifically mutated by an unnatural amino acid; wherein the unnatural amino acid is preferably pAcF, AeF, PrpF, NAEK or TetF;
the mutation on SpCas9 is at one or the following sites: positions K3, D39, H41, H116, D576, E945, K1151 and G1367 of SEQ ID NO. 1.

In an example of the present application, the present application provides a CRISPR family protein site-specifically mutated by an unnatural amino acid; wherein the unnatural amino acid is preferably pAcF, AeF, PrpF, NAEK or TetF;
the mutation on AsCas12a is at one or more of the following sites: positions M806, L834, S835, K860, K1086 of SEQ ID NO. 3.

In some embodiments of the present application, the present application further provides a CRISPR family protein site-specifically mutated by an unnatural amino acid, which is different from the wild-type CRISPR family protein in that the amino acid at position N of the sequence set forth in SEQ ID NO. 1 and SEQ ID NO. 3 is mutated to AeF, and the mutated amino acid residue is connected to the wild-type protein sequence in a mode as follows:

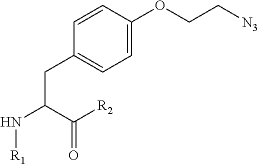

From R₁ to R₂ is the amino acid sequence of the protein from N terminus to C terminus, where the amino acid at position N is the selected site, including the amino acid at one of positions 3, 39, 41, 116, 576, 945, 1151 and 1367 of SpCas9; or the amino acid at one of positions 806, 834, 835, 860 and 1086 of AsCas12a; R₁ is the amino acid residue at position N−1, and R₂ is the amino acid residue at position N+1.

In some embodiments of the present application, the present application further provides a CRISPR family protein site-specifically mutated by an unnatural amino acid, which is different from the wild-type CRISPR family protein in that the amino acid at position N of the sequence set forth in SEQ ID NO. 1 and SEQ ID NO. 3 is mutated to NAEK, and the mutated amino acid residue is connected to the wild-type protein sequence in the following way:

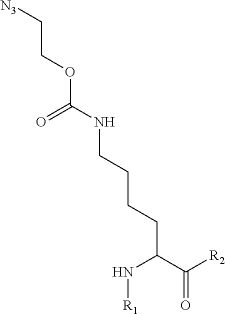

From R₁ to R₂ is the amino acid sequence of the protein from N terminus to C terminus, where the amino acid at position N is the selected site, including the amino acid at one of positions 3, 39, 41, 116, 576, 945, 1151 and 1367 of SpCas9; or the amino acid at one of positions 806, 834, 835, 860 and 1086 of AsCas12a; R₁ is the amino acid residue at position N−1, and R₂ is the amino acid residue at position N+1.

In an embodiment of the present application, SpCas9 has a molecular weight of 158.46 kDa, is consisted of 1368 amino acid residues, and has an amino acid sequence of SEQ ID NO. 1. The base sequence encoding SpCas9 is SEQ ID NO. 2. AsCas12a has a molecular weight of 143.56 kDa, is consisted of 1227 amino acid residues, and has an amino acid sequence of SEQ ID NO. 3. The base sequence encoding AsCas12a is SEQ ID NO. 4. AsCas12-BE has a molecular weight of 188.78 kDa, is consisted of 1638 amino acid residues, and has an amino acid sequence of SEQ ID NO. 6. The base sequence encoding AsCas12-BE is SEQ ID NO. 7.

In some embodiments of the present application, the present application further provides a site-specific nucleic acid conjugate of a CRISPR family protein, in which the CRISPR family protein is a CRISPR family protein site-specifically mutated by an unnatural amino acid described above, and the unnatural amino acid bears a group such as an azide, an alkynyl, an aldehyde, a ketone, a cyclopropene or a tetrazine group;
here, the nucleic acid bears a dibenzocyclooctyne (DBCO) or a Nor, BCN, TCO or sTCO group as follows:

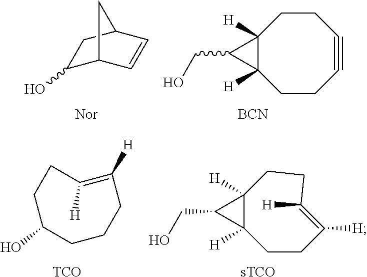

the unnatural amino acid undergoes an orthogonal chemical reaction with the nucleic acid described above, thereby resulting in a site-specific nucleic acid conjugate of the CRISPR family protein.

In some examples of the present application, the present application provides a site-specific nucleic acid conjugate of a CRISPR family protein, wherein the site-specifically mutated CRISPR family protein is conjugated to a DBCO modified nucleic acid in a way as follows:

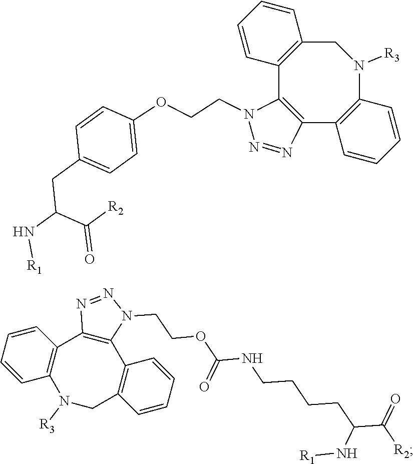

here, R₃ is a DNA or RNA of a different sequence;

from R₁ to R₂ is the amino acid sequence of the protein from N terminus to C terminus, where the amino acid at position N is the selected site, including the amino acid at one of positions 3, 39, 41, 116, 576, 945, 1151 and 1367 of SpCas9; or the amino acid at one of positions 806, 834, 835, 860 and 1086 of AsCas12a; R₁ is the amino acid residue at position N−1, and R₂ is the amino acid residue at position N+1.

In some embodiments of the present application, the nucleic acid in the site-specific nucleic acid conjugate of the CRISPR family protein may be an oligo DNA, a donor DNA, or a crRNA and a related modified nucleic acid thereof.

In some embodiments of the present application, the present application further provides a method for preparing the CRISPR family protein site-specifically mutated by an unnatural amino acid or the site-specific nucleic acid conjugate thereof described above. The preparation method includes the steps of:
(1) site selection: based on structural information of the CRISPR family protein such as SpCas9 or AsCas12a, selecting one or more specific amino acid sites in the amino acid sequence of the CRISPR family protein;
(2) genetic mutagenesis: mutating the codon encoding the amino acid at the site selected in step (1) to codon TAG by a genetic engineering method, to obtain a mutated CRISPR protein gene;
(3) construction of an expression vector: ligating the mutated CRISPR protein gene obtained by genetic engineering in step (2) to an expression plasmid by means of molecular cloning, to obtain a mutant expression vector plasmid comprising a mutant sequence;
(4) protein expression: co-transfecting the mutant expression vector plasmid obtained in (3) and a tool plasmid for unnatural amino acid (such as plasmid pUltra-MjPolyRS) into the same host cell, culturing the host cell successfully transfected under a condition where a host cell culture solution is supplemented with an unnatural amino acid such as pAcF, AeF, or NAEK, and inducing the expression of a mutant protein in the host cell;

Here, the tool plasmid for unnatural amino acid encodes tRNA and aminoacyl-tRNA synthetase, which can specifically recognize TAG and insert an unnatural amino acid such as AeF or NAEK at the position corresponding to this codon.

In some embodiments of the present application, the preparation method may further include:
for a host capable of expressing a mutant protein, evaluating the expression yield of the CRISPR protein and detecting its activity. A mutant capable of retaining more than 80% of the yield and activity of the wild-type is determined as a candidate for site-specific modification.

In some embodiments of the present application, the preparation method may further include:
expressing a mutant protein that meets the above requirements, and subjecting the product thereof to further purification; and
detecting the activity of the CRISPR family protein containing unnatural amino acid.

In some examples of the present application, the preparation method may further include:
adding DBCO-modified single-stranded donor DNA to SpCas9 protein containing unnatural amino acid, to make the azide group on the protein undergo click chemical reaction with DBCO on the DNA, and then detecting the in vitro and in vivo activities of SpCas9 protein;
adding DBCO modified adaptor-DNA to SpCas9 protein containing unnatural amino acid, to prepare a stable protein-nucleic acid adaptor complex, and then detecting the in vitro and in vivo activities thereof; and
to a mutant whose activity of SpCas9 is not affected, adding donor DNA having partial base-pairing with the adaptor, to have them combined into a RNP-adaptor-donor DNA complex, and transfected into cells. A SpCas9-ssODN complex with improved efficiency of homologous recombination repair than the wild-type is obtained.

In some examples of the present application, the preparation method may further include:
adding DBCO-modified crRNA to AsCas12a protein containing unnatural amino acid, wherein after the protein and crRNA form the correct binding conformation through affinity, the unnatural amino acid containing azide group, which is in the vicinity of DBCO modification, undergoes click chemical reaction through proximity effect to make them form a covalent RNP complex; and then detecting the in vitro activity thereof; and
a mutant whose activity of AsCas12a is not affected is transfected into cells. A modified AsCas12a protein, i.e., the AsCas12a mutant protein with improved efficiency of gene cleavage than the wide-type is obtained.

In the above examples, the DBCO-modified adaptor DNA may be of a nucleic acid sequence for any target region of DNA, with a length of about 18-30 bases, and is complementarily paired with the homologous arms of the corresponding donor DNA, to incorporate DBCO modification at 3′ end of the donor DNA through a chemical reaction.

In the above examples, the donor DNA may be recruited by complementary base pairing at the 5′ end of the adaptor DNA.

In the above examples, the DBCO modified crRNA and the improved crRNA may be of nucleic acid sequences for any target region of DNA, to incorporate DBCO modification at the 5′ end of target region of DNA.

In some embodiments of the present application, the present application provides a corresponding nucleic acid sequence encoding the CRISPR family protein site-specifically mutated by an unnatural amino acid described above, among which, for example, the base sequence for encoding SpCas9 is SEQ ID NO. 2, and the codon corresponding to the mutated amino acid in the base sequence is TAG, TAA or TGA, preferably TAG; for example, the base sequence for encoding AsCas12a is SEQ ID NO. 4, and the codon corresponding to the mutated amino acid in the base sequence is TAG, TAA or TGA, preferably TAG.

In some embodiments of the present application, the present application further provides a vector for preparing the CRISPR family protein site-specifically mutated by an unnatural amino acid or the nucleic acid conjugate thereof described above, and the vector contains the corresponding nucleic acid sequence encoding the CRISPR family protein site-specifically mutated by an unnatural amino acid described above.

In some examples of the present application, plasmids used to prepare the CRISPR family protein site-specifically mutated by an unnatural amino acid or the nucleic acid conjugate thereof described above include, but are not limited to, pET28a-Cas9-K3, pET28a-Cas9-D39, pET28a-Cas9-H116, pET28a-Cas9-H41, pET28a-Cas9-D576, pET28a-Cas9-E945, pET28a-Cas9-K1151, or pET28a-Cas9-G1367; pET22b-Cas12a-M806, pET22b-Cas12a-K1086.

In some embodiments of the present application, the present application further provides a host cell for preparing the CRISPR family protein site-specifically mutated by an unnatural amino acid or the nucleic acid conjugate thereof described above, the host cell contains a helper plasmid and a tool plasmid for unnatural amino acid described above, here, the helper plasmid is a plasmid for expressing tRNA^{tyr} and aminoacyl-tRNA synthetase; optionally, the host cell is a eukaryotic host cell or a prokaryotic host cell.

In an embodiment of the present application, the present application provides uses of the CRISPR family protein site-specifically mutated by an unnatural amino acid described above or the site-specific nucleic acid conjugate thereof, including but without limitation, e.g., use in improving efficiency of gene editing, improving efficiency of homologous recombination, improving efficiency of base editing, and applications in improving gene editing efficiency in mouse embryonic stem cells.

In the present application, an unnatural amino acid is introduced into a CRISPR family protein. Owing to the presence of this unnatural amino acid, a chemically modified nucleic acid may be anchored to the unnatural amino acid for site-specific protein modification through click chemical reaction, rather than other positions. This is essential for ensuring the functional integrity of this CRISPR family protein.

Specifically, in one specific embodiment of the present application, the CRISPR family protein introduced with an unnatural amino acid is provided, it is mainly through two steps:
1) constructing a vector encoding the CRIPSR family protein whose amino acid at a selected amino acid site is mutated to TAG; and
2) co-expressing a tool plasmid for unnatural amino acid which is required, with a plasmid constructed in step 1) in a suitable host bacterium, and adding the desired unnatural amino acid to culture medium in order to obtain the CRISPR family protein introduced with an unnatural amino acid.

In one specific embodiment of the present application, SpCas9 or AsCas12a gene with an amber codon TAG is inserted into a vector (pET28a-Cas9 plasmid, pET22b-Cas12a plasmid, pET22b-Cas12a-BE plasmid), and the expression plasmid is transformed into Escherichia coli engineering bacteria together with a tool plasmid for unnatural amino acid, and the site-specifically mutated SpCas9 or AsCas12a protein is obtained by adding an unnatural amino acid such as pAcF, AeF or NAEK into the culture solution.

In one specific embodiment of the application, the present application provides a SpCas9 protein with a site-specifically introduced unnatural amino acid containing azide group. The site-specifically mutated protein may undergo click chemical reaction with DBCO modified oligo DNA to anchor the oligo-DNA at a specific position of the SpCas9 protein. This DNA can recruit donor DNA to the vicinity of Cas9 through complementary base pairing to form an RNP-oligo DNA-donor DNA complex, which can increase the concentration of donor DNA in the vicinity of DSB during delivery, and improve in vivo efficiency of homologous recombination repair.

In one embodiment of the present application, the present application provides an AsCas12a protein with a site-specifically introduced unnatural amino acid containing azide group. The azide group on the site-specifically mutated protein may undergo click chemical reaction with the DBCO modified crRNA, to anchor the crRNA onto the AsCas12a protein to form an RNP complex. The crRNA and the AsCas12a protein in the RNP complex will not dissociate from each other during delivery, thus will not cause the concentration of RNP to be insufficient at the target region of DNA, which can improve the efficiency of in vivo cleavage, efficiency of homologous recombination and efficiency of base editing.

Provided in the present application is a CRISPR family protein which can be recombinantly expressed in E. coli and has a site-specifically introduced unnatural amino acid mutation, and the introduction of this site-specific mutation does not affect the in vitro and in vivo functions of the CRISPR family protein. At the same time, the in vivo and in vitro cleavage activities of the SpCas9 protein which is conjugated to oligo DNA and recruits donor DNA are not affected; the in vitro cleavage activity of AsCas12a conjugated to crRNA is not affected, which when delivered to a cell, improve the efficiency of cleavage, efficiency of homologous recombination and efficiency of base editing.

The present application provides a method for site-specifically covalently anchoring a nucleic acid. In this method, a protein with a site-specifically introduced reactive group, and a chemically modified nucleic acid, are covalently conjugated through bioorthogonal reactions. The bioorthogonal reactions include, but are not limited to, 1,3-dipolar cycloaddition reaction (also known as copper-free click chemistry) between azide and DBCO, Diels-Alder reaction between tetrazine and cyclic olefin or cyclic alkyne.

In the present application, the technologies of gene codon expansion used may refer to the technology of inserting synthetic unnatural amino acids into proteins proposed by Professor Peter Schultz in 2001. All proteins in nature are composed of 20 kinds of amino acids encoded by 61 codons, among which three stop codons, TAG, TAA and TGA, perform the termination of protein translation. The key to this technology is to change the biological function of TGA and TAG, and insert the “artificial amino acid” artificially synthesized with special physical and chemical characteristics into any selected site of any selected protein. With these artificial amino acids, a protein may be site-specifically introduced with functional groups with diverse functions and special chemical, physical or biological activities, for example, special chemical groups such as carbonyl, alkynyl and azide groups. These groups may effectively and selectively form covalent bonds, which are more conducive to site-specific modification of the protein, thereby improving properties of the protein or enhancing the functions thereof. Up to date, this technology has successfully made dozens of kinds of unnatural amino acids site-specifically being introduced into proteins expressed in living cell, giving these proteins new physical, chemical and physiological properties. This site-specific modification differs from random modification which has no quality control standard and from the above-mentioned three site-specific modifications which require addition of a specific amino acid sequence to the modified protein.

For the concept of “bioorthogonal reaction”, see Hang, H. C., Yu, C., Kato, D. L., and Bertozzi, C. R. (2003). A metabolic labeling approach toward proteomic analysis of mucin-type O-linked glycosylation. Proceeding of the National Academy of Sciences of the United States of America 100, 14846-14851. This reaction refers to a chemical reaction capable of occurring in a biological system without interfering with endogenous biochemical processes. Compared with traditional labeling methods, such as fluorescent protein labeling, the advantages of using small chemical molecules as labels are that they are easier to enter into cells and have high selectivity. Bioorthogonal reactions make it possible to investigate biomolecules (such as sugars, proteins and lipids) in organisms in real time. Through efforts for more than a decade, a series of bioorthogonal reactions have been widely used in fields, such as cell labeling, antibody modification, high-throughput sequencing, proteomics. To date, a vast variety of chemical conjugation strategies have been developed to meet bioorthogonality, such as the 1,3-dipolar cycloaddition reaction (also known as copper-free click chemistry) between azide compounds and cycloalkynes (Baskin, J. M., Prescher, J. A., Laughlin, S. T., Agard, N. J., Chang, P. V., Miller, I. A., Lo, A., Codelli, J. A., and Bertozzi, C. R. (2007). Copper-free click chemistry for dynamic in vivo imaging. Proceedings of the National Academy of Sciences 104, 16793-16797), the reaction of nitrone with cycloalkyne (Ning, X., Temming, R. P., Dommerholt, J., Guo, J., Ania, D. B., Debets, M. F., Wolfert, M. A., Boons, G.-J., and van Delft, F. L (2010). Protein Modification by Strain-Promoted Alkyne-Nitrone Cycloaddition. Angewandte Chemie International Edition 49, 3065-3068), the reaction of aldehyde or ketone to form oxime or hydrazone, Diels-Alder reaction between tetrazine and cyclic olefin or cyclic alkyne (Blackman, M. L., Royzen, M., and Fox, J. M. (2008). Tetrazine ligation: Fast Bioconjugation Based on Inverse-Electron-Demand Diels-Alder Reactivity. Journal of the American Chemical Society 130, 13518-13519), click reaction based on isocyanide (Stockmann, H., Neves, A. A., Stairs, S., Brindle, K. M., and Leeper, F. J. (2011). Exploring isonitrile-based click chemistry for ligation with biomolecules. Organic & biomolecular chemistry 9), and the quadricyclane coupling reaction (Sletten, E. M., and Bertozzi, C. R. (2011). A Bioorthogonal Quadricyclane Ligation. Journal of the American Chemical Society 133, 17570-17573). The above references are incorporated in their entirety into the present application by reference.

### EXAMPLES

To further clarify the purpose, technical solutions and advantages of the present application, examples of the present application will be described in detail below. It should be noted that the following examples of the present application and the features of the examples may be arbitrarily combined with each other unless there is a conflict.

Reagents used in Examples include the following:

Phanta Super-Fidelity DNA Polymerase (Vazyme), KOD OneTM PCR Master Mix (TOYOBO), DpnI (New England Biolabs, NEB), spectinomycin (Sigma), kanamycin (Sigma), ampicillin (Sigma), DMEM medium (Macgene), 1×Trypsin-EDTA 0.25% (Macgene), 1×PBS (Macgene), nickel medium (GE healthcare)

Instruments used in Examples include the following:

PCR machine (Biorad), electrophoresis apparatus (Tanon), Tanon gel imaging system (Tanon), ultrasonic processor (SONICS), autoclave (STIK), pure water apparatus (Millipore), Nanodrop (Thermo), benchtop centrifuge (Thermo), sub-ultracentrifuge (Beckman), AKTA protein purification system (GE healthcare), flow cytometer (Beckman CytoFLEX), cell incubator (Thermo)

### Example 1: Construction of Genetic Vectors Containing Site-Specifically Mutated SpCas9 and AsCas12a

### (1) Acquirement of Helper Plasmid

PUltra-MjPolyRS (purchased from Addgene Company) (hereinafter abbreviated as helper plasmid), can be used to express tRNA and tRNA synthetase for specific recognition and insertion of unnatural amino acid AeF.

### (2) Acquirement of Plasmids Containing SpCas9 or AsCas12a

PET28a-SpCas9 plasmid (Liang, Z., Chen, K., Li, T., Zhang, Y., Wang, Y., Zhao, Q., Liu, J., Zhang, H., Liu, C., Ran, Y., et al. (2017). Efficient DNA-free genome editing of bread wheat using CRISPR/Cas9 ribonucleoprotein complexes. Nature communications 8, 14261) can be used to express SpCas9 protein recombinantly in Escherichia coli. The sequence encoding the protein AsCas12a in pET22b-AsCas12a plasmid was cloned by PCR from Addgene plasmid #102565, with 5′ Nde1 and 3′ Xho1 cleavage sites being introduced at the ends by PCR primers. Then the sequence encoding the protein AsCas12a and the complete plasmid sequence was cleaved by restriction enzymes and ligated to construct the plasmid, and the complete sequence of pET22b-AsCas12a plasmid is obtained. The AsCas12a protein could be expressed recombinantly in Escherichia coli.

Sequences of the primers used are as follows:

Cas12a-2NLS-FW
GGGAATTGTGAGCGGATAACAATTCCCC
SV40-RV
CCCACTTTACGTTTCTTTTTAGGACTGCCCTTTTTCTTTTTTGCCTGGCC
GG
Cas12a-2NLS-RV
CAGTGGTGGTGGTGGTGGTGCTCGAGGGATCCCACTTTACGTTTCTTTTT
AGGACTGC

### (3) Selection of Sites for Site-Specific Mutagenesis and Construction of Mutant Plasmids.

Based on an analysis of results from resolution of crystal structure of SpCas9 protein, the inventors selected amino acids at positions 3, 39, 41, 116, 576, 945, 1151 and 1367 as the target amino acids for site-specific mutagenesis, and replaced these specific amino acid residues with an unnatural amino acid containing azide group. Then the resulted mutated SpCas9 proteins were used as raw materials for site-specific conjugation of SpCas9 to donor DNA, to make them together with gRNA form a RNP-DNA complex. When delivered to a position for a target region of cellular DNA, the donor DNA could be maintained in a sufficient concentration, thus improving the efficiency of homologous recombination repair.

Based on an analysis of results from resolution of crystal structure of AsCas12a protein, the inventors selected amino acids at positions M806 and K1086 as the target amino acids for site-specific mutagenesis, and replaced these specific amino acid residues with an unnatural amino acid containing azide group. Then the resulted mutated AsCas12a proteins were used as raw materials for site-specific conjugation of AsCas12a to crRNA, to make them form a covalent complex, thereby solving the problem of low cleavage efficiency caused by an insufficient RNP concentration in the vicinity of the target region of DNA due to dissociation of AsCas12a from crRNA during delivery.

For the two proteins SpCas9 and AsCas12a above, the inventors designed primers capable of mutating the corresponding codons encoding the amino acids at positions 3, 39, 41, 116, 576, 945, 1151 and 1367 of SpCas9, and positions M806 and K1086 of AsCas12a, respectively to TAG. The specific primers are shown in the following table.

TABLE 1
List of Mutant Primers

| Mutation | | |
|---|---|---|
| site | Primer | Sequence (5′-3′ direction) |
| K3 | K3TAG-FW | CCAAGAAGAAGAGGAAGGTGATGGATtAGAAATACTCAAT |
| | | AGGCTTAGATATCGGCAC |
| | K3TAG-RV | GTGCCGATATCTAAGCCTATTGAGTATTTCTaATCCATCACC |
| | | TTCCTCTTCTTCTTGG |
| D39 | D39TAG-FW | GTTCAAGGTTCTGGGAAATACATAGCGCCACAGTATCAAAA |
| | | AAAATC |
| | D39TAG-RV | GATTTTTTTTGATACTGTGGCGCTATGTATTTCCCAGAACCT |
| | | TGAAC |
| H41 | H41TAG-FW | GTTCTGGGAAATACAGACCGCTAGAGTATCAAAAAAAATCT |
| | | TATAGGGGCTC |
| | H41TAG-RV | GAGCCCCTATAAGATTTTTTTTGATACTCTAGCGGTCTGTAT |
| | | TTCCCAGAAC |
| H116 | H116TAG-FW | GAAGAAGACAAGAAGCATGAACGTTAGCCTATTTTTGGAA |
| | | ATATAGTAGATGAAGTTGC |
| | H116TAG-RV | GCAACTTCATCTACTATATTTCCAAAAATAGGCTAACGTTCA |
| | | TGCTTCTTGTCTTCTTC |
| K1151 | K1151TAG-F | CTAGTGGTTGCTAAGGTGGAATAGGGGAAATCGAAGAAGT |
| | | TAAA |
| | K1151TAG-R | TTTAACTTCTTCGATTTCCCCTATTCCACCTTAGCAACCACTA |
| | | G |
| D576 | D576TAG-FW | GAAGATTATTTCAAAAAAATAGAATGTTTTtagAGTGTTGAA |
| | | ATTTCAGGAGTTGAAG |
| | D576TAG-RV | CTTCAACTCCTGAAATTTCAACACTCTAAAAACATTCTATTTT |
| | | TTTGAAATAATCTTC |
| E945 | E945TAG-FW | GATAGTCGCATGAATACTAAATACGATtagAATGATAAACTT |
| | | ATTCGAGAGGTTAAAG |
| | E945TAG-RV | CTTTAACCTCTCGAATAAGTTTATCATTCTAATCGTATTTAGT |
| | | ATTCATGCGACTATC |
| G1367 | G1367TAG-FW | CGCATTGATTTGAGTCAGCTAGGATAGGACCCCAAGAAGA |
| | | AGAGGAAGGTG |
| | G1367TAG-RV | CACCTTCCTCTTCTTCTTGGGGTCCTATCCTAGCTGACTCAA |
| | | ATCAATGCG |
| M806 | M806TAG-FW | ACCGGCTGGGAGAGAAGtagCTGAACAAGAAGCTGAAGG |
| | M806TAG-RV | CCTTCAGCTTCTTGTTCAGctaCTTCTCTCCCAGCCGGT |
| K1086 | K1086TAG-FW | GTGGACCCCTTCGTGTGGTAGACCATCAAGAATCACGAGA |
| | | G |
| | K1086TAG-RV | CTCTCGTGATTCTTGATGGTCTACCACACGAAGGGGTCCAC |

Amplification was performed by using the above primers and KOD enzyme and with pET28a-Cas9 plasmid as the template, and the obtained PCR products were digested by DpnI. DH5α strain was transformed with the resulting products from digestion, and the DH5α strain recovered from the transformation was spread on plates with corresponding antibiotics. The next day, single clones were picked for sequencing, to obtain plasmids of pET28a-Cas9-K3, pET28a-Cas9-D39, pET28a-Cas9-H41, pET28a-Cas9-H116, pET28a-Cas9-D576, pET28a-Cas9-E945, pET28a-Cas9-K1151, and pET28a-Cas9-G1367.

### (4) Construction of Expression Strain for Site-Specifically Mutated SpCas9

Two plasmids, the helper plasmid pULTRA-Ambrx (spectinomycin resistance) obtained in step (1) and the expression plasmid pET28a-Cas9-G1367 (kanamycin resistance) obtained in step (3), were simultaneously transformed into E. coli BL21(DE3) subtype. Positive strains transformed with both plasmids was screened out by double resistance plates (spectinomycin resistance and kanamycin resistance) and named as expression strain pET28a-Cas9-G1367.

According to the same method, expression strains for other mutation sites were obtained, namely, expression strains pET28a-Cas9-K3, pET28a-Cas9-D39, pET28a-Cas9-H41, pET28a-Cas9-H116, pET28a-Cas9-D576, pET28a-Cas9-E945, and pET28a-Cas9-K1151.

### Example 2: Expression and Purification of SpCas9 with Point Mutation

1: Amino Acids were Purchased Commercially
2: Expression of SpCas9 Protein with Site-Specific Insertion of Unnatural Amino Acid AeF

The expression strain pET28a-Cas9-G1367 obtained in step (4) of Example 1 was cultured in 2YT medium (containing 100 ug/ul spectinomycin and 100 ug/ul kanamycin) at 37° C. overnight. The next day, the resultant culture was inoculated into fresh culture medium at 1:100, and cultured. When OD value reached 0.3, 1 mM AeF was added and culturing was continued. When OD value was 0.6-1.0, 0.2 mM isopropyl β-D-1-thiogalactopyranoside (IPTG) was added and the culture was incubated at 18-20° C. for 16-18 hours to induce expression, and then bacteria were collected. The positive control used in this expression experiment was the wild-type pET22b-Cas9 expression strain (obtained in step 2 of Example 1), and the conditions were the same as those of the mutant strain except for different strain being inoculated.

Other mutant expression strains were all expressed according to the above conditions.

### 3: Purification of AeF Mutant Protein

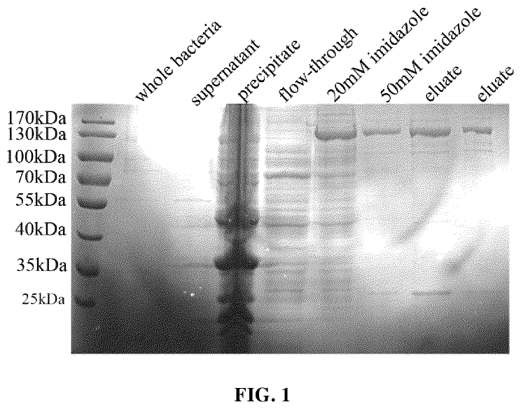

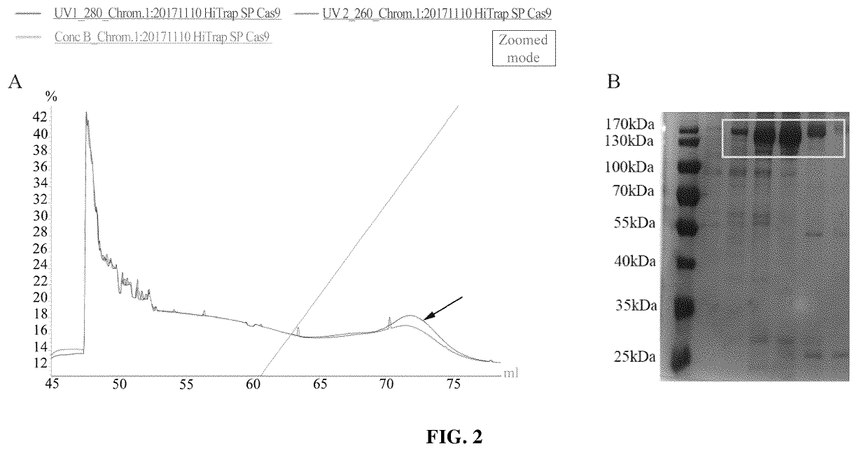

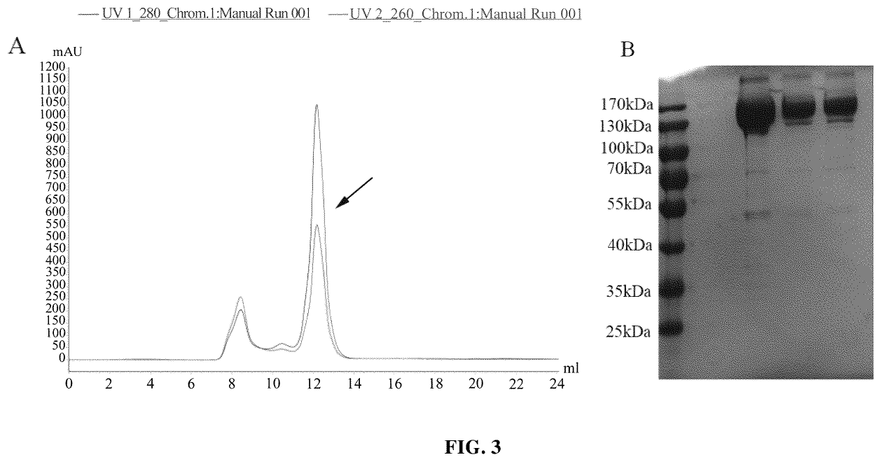

1) the collected expression bacteria mentioned above was resuspended in a high-salt buffer (20 mM Tris, pH8.0, 1M KCl), and disrupted by ultrasonication.
2) the disrupted product of step 1) was subjected to high speed centrifugation, and the supernatant was pipetted as a soluble component.
3) the product of step 2), i.e., the supernatant sample, was purified with Ni affinity column and eluted with high concentration imidazole. The results are shown in FIG. 1.
4) the purified product of step 3) was exchanged into a low-salt buffer (20 mM Tris, pH8.0, 300 mM KCl), and further purified by cation exchange. The elution buffer was 20 mM Tris, pH8.0, 1M KCl. Target protein fractions were collected. The results are shown in FIG. 2.
5) the purified product of step 4) was further purified by molecular sieve chromatography, using Superdex 200 (GE healthcare) column, with an elution buffer of 20 mM Tris, pH8.0, 150 mM KCl. Target protein fractions were collected. The results are shown in FIG. 3.

After purification by the above three processes, the electrophoresis pure grade (>95%) target protein samples can be successfully obtained (FIG. 3), providing enough experimental samples for subsequent activity verification and function exploration.

4: Purification of other AeF Mutant Proteins

The expression strains pET28a-Cas9-K3, pET28a-Cas9-D39, pET28a-Cas9-H41, pET28a-Cas9-D576, pET28a-Cas9-E945, pET28a-Cas9-K1151 were cultured, and mutant proteins were purified and isolated, according to the above steps 2-3, to obtain purified products of the mutant proteins in which amino acids at positions 3, 39, 41, 116, 576, 945, 1151 and 1367 of SpCas9 were site-specifically mutated by unnatural amino acid AeF, and which were abbreviated as K3AeF-Cas9, D39AeF-Cas9, H41AeF-Cas9, H116AeF-Cas9, D576AeF-Cas9, E945AeF-Cas9, K1151AeF-Cas9, and G1367AeF -Cas9.

5: Expression and Purification of SpCas9 Protein Site-Specifically Inserted with Unnatural Amino Acid NAEK
1) the unnatural amino acid NAEK was purchased from commercial directly.
2) SpCas9 with NAEK insertion at respective sites was expressed and purified under conditions were the same as those in the above-mentioned steps 2-4, except that AeF was replaced by NAEK and MjPolyRS was replaced by WT PylRS. Purified products of the mutant proteins in which the amino acids at positions 3, 39, 41, 116, 576, 945, 1151 and 1367 of SpCas9 was site-specifically mutated by unnatural amino acid NAEK, and which were abbreviated as K3NAEK-Cas9, D39NAEK-Cas9, H41NAEK-Cas9, H116NAEK-Cas9, D576NAEK-Cas9, E945NAEK-Cas9, K1151NAEK-Cas9, and G1367NAEK-Cas9 were obtained.

### 6: Verification of Activities of Mutant SpCas9s

In order to verify whether SpCas9 and AsCas12a inserted with an unnatural amino acid have the function of cutting double-stranded DNA under the guide of gRNA while being expressed with high efficiency, the inventors tested the activities thereof. SpCas9 with the unnatural amino acid AeF inserted at position 1367 was used as an example, and was subjected to activity detection. Detection methods for mutants at other individual positions were similar.

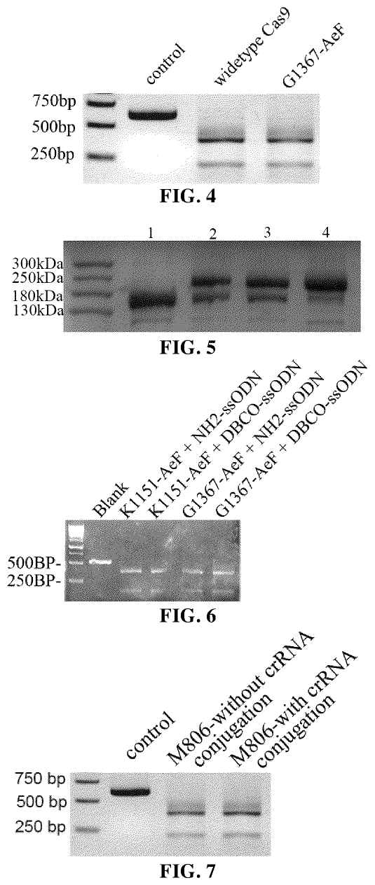

3 pmol of proteins (wild type and mutant type) and 9 pmol of gRNA were incubated at room temperature for 10 minutes to form RNP complexes. The template double-stranded DNA (100 μg) prepared in advance and the RNP were added into a reaction system containing NEB3.1 buffer, incubated at 37° C. for 1 hour and thermally inactivated under the condition of 65° C. Subsequently, the reaction products were used for nucleic acid gel separation. The results are shown in FIG. 4, demonstrating that the mutated SpCas9s and AsCas12a had the same cleavage activity as the wild type. The results show that the insertion of unnatural amino acids does not affect the recognition and cleavage activity of the protein to double-stranded DNA.

Sequences of gRNA and template DNA amplification primers used are as follows:

SpCas9-gRNA
GGGCGAGGAGCTGTTCACCGGTTTTAGAGCTAGAAATAGCAAGTTAAAAT
AAGGCTAGTCCGTTATCA
ACTTGAAAAAGTGGCACCGAGTCGGTGC
Tem-Fw
TCCAAAATGTCGTAACAACTCCGCC
Tem-Rv
ATGTTGCCGTCCTCCTTGAAGTC

Template DNA(SEQ ID NO.5) sequence is as follows:

TCCAAAATGTCGTAACAACTCCGCCCCATTGACGCAAATGGGCGGTAGGC
GTGTACGGTGGGAGGTCTATATAAGCAGAGCTCTCTGGCTAACTAGAGAA
CCCACTGCTTACTGGCTTATCGAAATTAATACGACTCACTATAGGGAGAC
CCAAGCTGGCTAGCGTTTAAACGGGCCCTCTAGACTCGAGATGGTGAGCA
AGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGC
TGCCCGTGCCCTGGCCCACCCTCGTGACCACCCTGAgCcACGGCGTcCAG
TGCTTCAGCCGCTACCCCGACCACATGAAGCAGCACGACTTCTTCAAGTC
CGCCATGCCCGAAGGCTACGTCCAGGAGCGCACCATCTTCTTCAAGGACG
ACGGCAACTACAAGACCCGCGCCGAGGTGAAGTTCGAGGGCGACACCCTG
GTGAACCGCATCGAGCTGAAGGGCATCGACTTCAAGGAGGACGGCAACA
T.

### Example 3: Covalent Conjugation of Mutants to DBCO Modified Nucleic Acid Molecules

After SpCas9 and AsCas12a were incorporated with an unnatural amino acid containing azide group, they can be conjugated to a DBCO-modified nucleic acid molecule by means of click chemistry reaction. This conjugation method was tested hereafter.

### 1) Synthesis of DBCO Modified Nucleic Acids

DBCO-modified single-stranded DNA or RNA were commercial products, and were purchased from Anhui General Biosynthesis Company and American IDT Company (Integrated DNA Technologies, Inc).

Sequences were as follows:

DBCO modified DNA
CAAATTCGTTGTCATACCTAGAAGA
DBCO modified AsCas12a-crRNA
GGGCGAGGAGCTGTTCACCGGTTTTAGAGCTATGCT

### 2) Conjugation to Nucleic Acids by Click Chemical Reaction

The mutated proteins and the DBCO modified nucleic acids were mixed to uniformity in a ratio of 1:1. Reactions were performed under conditions such as room temperature and 4° C. to explore reaction conditions. Samples were taken at regular intervals, and identified by SDS-PAGE. The results are shown in FIG. 5. It can be seen from the figure that the click chemical reaction was in a relatively fast speed and the equilibrium can be reached within 3 hours at 4° C., while reaction for a long time would greatly increase protein degradation. According to the experimental results, the reaction time was determined to be 3 hours, where the nucleic acids can be conjugated to about 95% of proteins.

The method for conjugating the mutant proteins having AeF or NAEK at other sites to DBCO modified nucleic acids was the same as above. Conjugates with approximate yields can be obtained.

### Example 4: Evaluation of In Vitro Activity of CRISPR Family Protein Site-Specifically Modified by a Nucleic Acid

1) 3 pmol of above-mentioned mutant protein SpCas9-G1367-AeF conjugated to single-stranded DNA (wild type protein as the control) and 9 pmol of gRNA were incubated at room temperature for 10 minutes to form a RNP complex. The template double-stranded DNA (100 μg) prepared in advance and the RNP were added into a reaction system containing NEB3.1 buffer, incubated at 37° C. for 1 hour and thermally inactivated under the condition of 65° C. Subsequently, the reaction products were used for nucleic acid gel separation. The results are shown in FIG. 6, demonstrating that the SpCas9 conjugated to single-stranded DNA has the same cleavage activity as the wild type. The results showed that the insertion of unnatural amino acids and conjugation of single-stranded DNA does not affect the recognition and cleavage activity of the proteins to double-stranded DNA.

Method for detecting in vitro activity of other mutant proteins inserted with AeF and NAEK and conjugated to single-stranded DNA was the same as that mentioned above. Approximate cleavage efficiency can be obtained.
2) 3 pmol of mutant protein AsCas12a-M806-AeF reacted with 3 pmol of DBCO modified crRNA at 4° C. for 3 hours (control group was wild type protein incubated with crRNA at room temperature for 10 minutes) to form a RNP complex, and the template double-stranded DNA (100 μg) prepared in advance and the RNP were added into a reaction system containing NEB3 .1 buffer, incubated at 37° C. for 1 hour and thermally inactivated in the condition of 65° C. Subsequently, the reaction products were used for nucleic acid gel separation. The results are shown in FIG. 7, demonstrating that the AsCas12a conjugated to crRNA has the same cleavage activity as the wild type. The results showed that the insertion of unnatural amino acids and conjugation of crRNA does not affect the recognition and cleavage activity of AsCas12a proteins to double-stranded DNA.

Method for detecting in vitro activity of other mutant proteins inserted with AeF and NAEK and conjugated to crRNA was the same as that mentioned above. Approximate cleavage efficiency can be obtained.

### Example 5: Evaluation of In Vivo Activity of CRISPR Family Protein Site-Specifically Modified by a Nucleic Acid

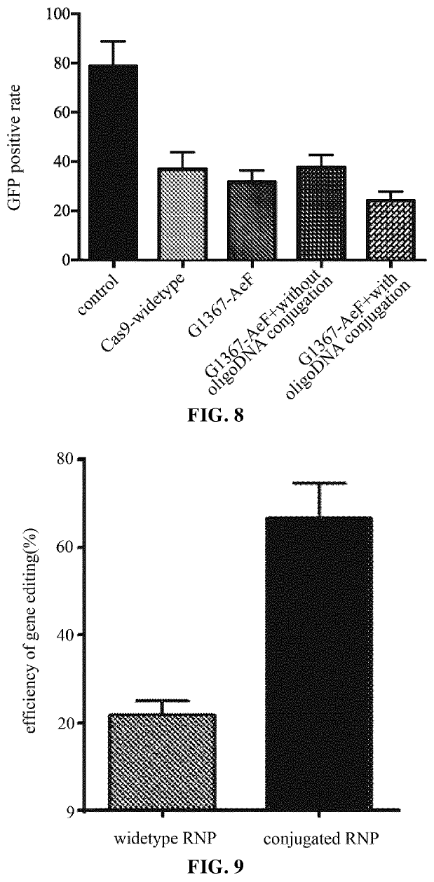

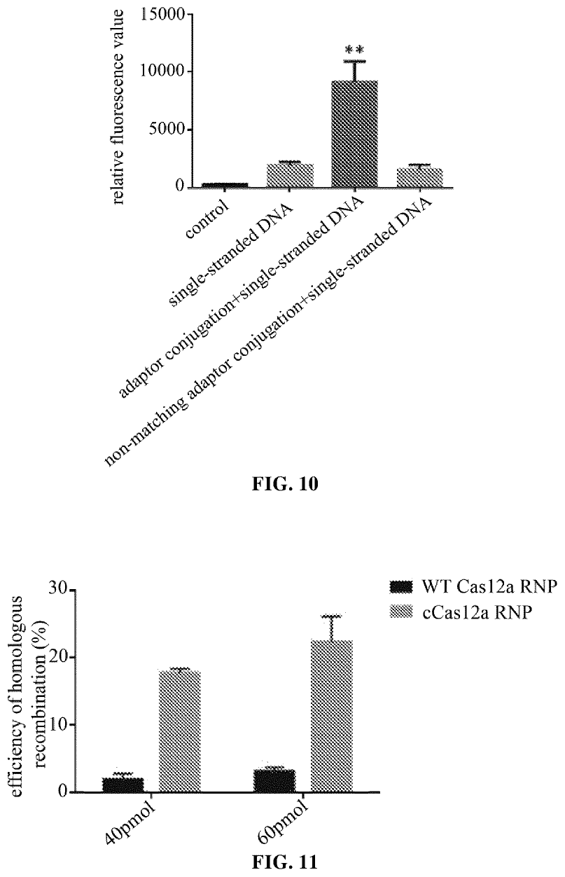

The specific process was as follows: firstly, the adherent reporter cell line was digested with trypsin, suspended in PBS and then counted. According to the counting result, 1×10⁶ cells were collected and suspended in 20 μL electroporation buffer (produced by Celetrix company, in which solution A and solution B were mixed in equal proportion before use). 18 pmol of RNP or nucleic acid conjugated RNP were gently mixed with cells, then placed in a specialized electroporation cuvette and subjected to electric shock according to optimized parameters (420V, 30 ms). After the electric shock, the transfected cells were quickly added into 2 ml preheated DMEM, and cultured in a six-well plate in a 37° C. incubator. After 24 hours, doxycycline hydrochloride solution was added to a final concentration of 10 ug/ml. After 48 hours, the cells were digested, suspended and exchanged into PBS buffer for flow cytometry analysis. The results are as shown in FIG. 8: the GFP fluorescence quenching rate of nucleic acid-conjugated SpCas9 in cells can substantially reach about 80-90% of the rate of the wild type; the combinations of wild-type AsCas12 and different RNAs can achieve an editing efficiency of about 20%, and the GFP fluorescence quenching rate of AsCas12a conjugated to crRNA in cells is 4-5 fold higher than that of the wild-type (FIG. 9), indicating that this conjugation of crRNA to AsCas12a protein can improve the cleavage efficiency.

The inventors thus constructed a reporter cell line evaluation system. The lentivirus system pLV was used to package lentivirus containing EGFP gene downstream of Teton promoter, and was used to infect 293T cells. Through flow sorting, cells with strongly positive green fluorescence were sorted to obtain a monoclonalized cell line. This system exploited fluorescence quenching of GFP protein for characterization, which was different from the cell line directly expressing GFP protein in that the expression of GFP in the reporter cell line of the inventors' was controlled by Teton operon, and it was inductively expressed only when Doxycycline (Dox) was added. The reporter cell line can greatly shorten the time to observe GFP fluorescence quenching. In this experiment, nucleic acid conjugated RNP was delivered into cells by electroporation, and subsequent transcription and translation of GFP gene were disturbed by RNP cutting, and thus the quenching rate of GFP was observed to detect the editing efficiency of RNP on gene.

### Example 6: Evaluation of Efficiency of Gene Editing by Site-Specifically oligoDNA Conjugated SpCas9 in Cells

In this Example, by cutting gene before TAG codon of conserved gene GAPDH in cells, a HiBiT gene encoding 13 amino acids was inserted through donor DNA, which can encode and express HiBiT in a fusion to GAPDH. HiBiT and LgBiT can form a complete nanoluciferase, releasing fluorescence with the participation of a substrate. Whether fluorescence was released or not was used to assess gene editing efficiency. For the transfection of RNP-Oligo DNA complex, 18 pmol of Cas9 wild type/mutant protein and 24 pmol of sgRNA were firstly mixed at room temperature and incubated for 5 minutes, then 18 pmol of nucleic acid matching Adaptor was added to the complex, and incubated for 3 hours at 4° C. Before transfection, 18 pmol of single-stranded donor DNA (ssODN) was added to the mixture for transfection in the next step. Transfection was carried out by CTX-1500A LE electroporator, and buffers A and B were prepared and mixed in an equal proportion according to the instructions. For a single reaction, 20 ul electroporation buffer was used to resuspend 1*10⁶ cells, and the pre-made conjugation complex was added into the cell suspension for electroporation where the electroporation condition is 420 V 30 ms. Immediately after electroporation, the cells were aspirated and placed into preheated DMEM medium to continue culturing for 48 hours. At 48 hours after transfection, the cells were collected and lysed, and fluorescence generation was detected by using the Nano-Glo® HiBiT Lytic Detection System kit provided by Promega Company, to evaluate the effect on gene editing efficiency after RNP-DNA conjugation. Results are as shown in FIG. 10 that oligo DNA conjugation to RNP can significantly improve the gene editing efficiency, demonstrating that the method of recruiting donor DNA by RNP with site-specific oligo DNA conjugation established by the inventors can indeed improve the efficiency of homologous recombination repair in gene editing.

The nucleic acid sequences used in this Example are shown in the following table:

sgRNA targeting GAPDH sequence
CCTCCAAGGAGTAAGACCCC
single-stranded donor DNA GAPDH-186 nt SSODN
TCTTCTAGGTATGACAACGAATTTGGCTACAGCAA
CAGGGTGGTGGACCTCATGGCCCACATGGCCTCCA
AGGAGGTGAGCGGCTGGCGGCTGTTCAAGAAGATT
AGCTAAGACCCCTGGACCACCAGCCCCAGCAAGA
GCACAAGAGGAAGAGAGAGACCCTCACTGCTGGG
GAGTCCCTGCCAC

### Example 7: Evaluation of the Level of Homologous Recombination by Site-Specifically crRNA Conjugated AsCas12a in Cells

In this example, a conserved gene in human cells, HPRT gene, was cut, and a 6 bp specific sequence, which is the EcoR1 specific recognition site, along with 40 bp homologous arms on the left and right sides was introduced at the cutting site. If the gene editing is successful and accurate homologous recombination occurs, corresponding fragments can be genome-specifically amplified, and in turn subjected to evaluation by fragment enzyme digestion. The efficiency of EcoR1 cutting can directly reflect the level of homologous recombination. Specifically, the procedure was as follows: 40/60 pmol AsCas12a protein and corresponding crRNA (1.2× equivalent) were incubated in vitro for 10 minutes to form RNP, then allowed to react at 4 degrees for 3 hours, and then 0.6× equivalent of single-stranded donor DNA was added to form complexes for subsequent transfection. Transfection was carried out using CTX-1500A LE electroporator, and buffers A and B were prepared and mixed in an equal proportion according to the instructions. For a single reaction, 20 ul electroporation buffer was used to resuspend 1*10⁶ cells, and the pre-made conjugation complex was added into the cell suspension for electroporation where the electroporation condition is 420 V 30 ms. Immediately after electroporation, the cells were aspirated and placed into preheated DMEM medium to continue culturing for 48 hours. 48 hours after transfection, the cells were collected, and the cell genome was extracted using a genome extraction kit provided by Vazyme company. After PCR amplification of the target region, the amplified products were purified by Cycle pure purification kit and then quantified. The evaluation of efficiency of homologous recombination was identified by enzyme digestion. The specific procedure was as follows: the sample was added in an amount of 200 ng PCR product/reaction, in an enzyme digestion system of 10 ul. 1 ul Cutsmart solution, 200 ng template and 1 ul EcoRI-HF enzyme were added, with water added up to 10 ul. Reaction occurred at 37 degrees for 1 hour, and the products were identified by 1% agarose gel electrophoresis. The bands were quantitatively analyzed. Results are as shown in FIG. 11. The conjugation group (cCas12a) was observed to be able to significantly improve the efficiency of homologous recombination by 7-8 fold at both 40 pmol and 60 pmol RNP cleavage concentration, demonstrating that the method of site-specific conjugating crRNA to RNP established by the inventors can indeed improve the efficiency of homologous recombination repair in gene editing.

The nucleic acid sequences used in this Example are shown in the following table:

crRNA targeting human HPRT sequence
GGUUAAAGAUGGUUAAAUGAU
ssDON used by HDR
ATAAGCCATTTCACATAAAACTCTTTTAGGTTAAAGATGGGAATTCTTA
AATGATTGACAAAAAAAGTAATTCACTTACAGTCTGG
PCR forward primer
CATGGTACACTCAGCACGGATGAAATG
PCR reverse primer
GCTGTTCAACTATTTCAGCCAACAAGAAGTG

### Example 8: Evaluation of Base Editing Efficiency of Site-Specifically crRNA Conjugated AsCas12a Base Editor in Cell

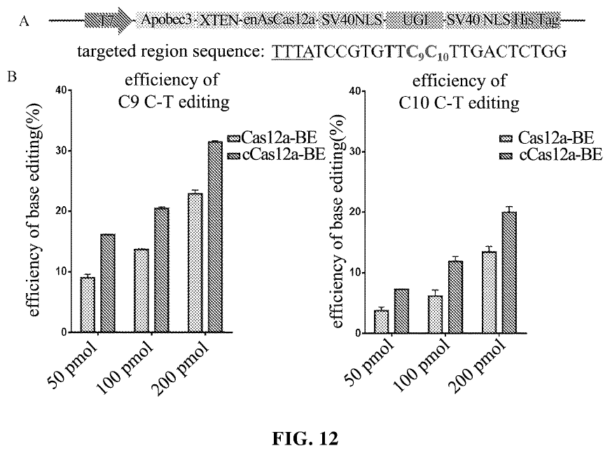

In this example, the protein containing AsCas12a base editor (AsCas12a-BE) were firstly expressed and purified. The mutation site on the mutant was M806AeF, and the plasmid construction and protein expression and purification were the same as those in Example 2 above. After the protein was obtained, the efficiency of base editing was evaluated at the cell level. The evaluation system used in this example edits the endogenous gene FANCF of human cell 293T. The specific procedure was as follows: 200 pmol AsCas12a-BE protein and corresponding crRNA (1.2× equivalent) were incubated in vitro for 10 minutes to form RNP, then allowed to react at 4 degrees for 3 hours to form complexes for subsequent transfection. Transfection was carried out by CTX-1500A LE electroporator, and buffers A and B were prepared and mixed in an equal proportion according to the instructions. For a single reaction, 20 ul electroporation buffer was used to resuspend 1*10⁶ cells, and the pre-made conjugation complex was added into the cell suspension for electroporation where the electroporation condition is 420 V 30 ms. Immediately after electroporation, the cells were aspirated and placed into preheated DMEM medium to continue culturing for 48 hours. 48 hours after transfection, the cells were collected, and the cell genome was extracted using the genome extraction kit provided by Vazyme company. The target region was amplified by PCR for sanger sequencing, and analyzed by the online EditR tool. As shown in FIG. 12, the efficiency of base editing by the conjugation group was significantly better than that of the wild-type protein at various concentrations, demonstrating that the method of site-specifically conjugating crRNA to RNP established by the inventors can indeed improve the base editing efficiency.

The nucleic acid sequences used in this Example are shown in the following table:

crRNA targeting human FANCF sequence
UCCGUGUUCCUUGACUCUGG
PCR forward primer
GAAGGCCCAGAATTCAGCATAGCGC
PCR reverse primer
GTCCCAGGTGCTGACGTAGGTAG

Although the embodiments disclosed in this application are as above, the contents described are only the embodiments used to facilitate the understanding of this application, and are not intended to limit this application. Any person skilled in the art to which this application belongs may make any modifications and changes to the form and details of implementation without departing from the spirit and scope disclosed in this application. However, the scope of patent protection of this application shall still be subject to the scope defined in the appended claims.

1. A method for site-specific conjugation of a nucleic acid to a CRISPR family protein, comprising:
(a) mutating the CRISPR family protein site-specifically with an unnatural amino acid; and
(b) conjugating the nucleic acid to the CRISPR family protein site-specifically mutated by the unnatural amino acid obtained in step (a), thereby obtaining a site-specific nucleic acid conjugate of the CRISPR family protein;
wherein the unnatural amino acid has orthogonal chemical reactivity.

2. The method for site-specific conjugation of claim 1, wherein the CRISPR family protein comprises, but is not limited to, Cas9 protein, Cas12a protein, CasX protein, Cas ϕ protein, Cas12g protein from different species or genera, or a related inactivated counterpart thereof having no cleavage activity but retaining binding activity.

3. The method for site-specific conjugation of claim 1, wherein the orthogonal chemical reactivity means, but is not limited to, that the unnatural amino acid comprises an azido, an alkynyl, a cyclopropenyl or a tetrazine group.

4. A CRISPR family protein site-specifically mutated by an unnatural amino acid, wherein the unnatural amino acid bears an azido, an alkynyl, a cyclopropenyl or a tetrazine group; the CRISPR family protein comprises, but is not limited to, Cas9 protein, Cas12a protein, CasX protein, Cas ϕ protein, Cas12g protein from different species or genera, or a related inactivated counterpart thereof having no cleavage activity but retaining binding activity.

5. The CRISPR family protein site-specifically mutated by an unnatural amino acid of claim 4, wherein the CRISPR family protein is SpCas9 mutated at one or more of: positions K3, D39, H41, H116, D576, E945, K1151 and G1367 of SEQ ID NO.1.

6. The CRISPR family protein site-specifically mutated by an unnatural amino acid of claim 4, wherein the CRISPR family protein is AsCas12a mutated at one or more of: positions M806, L834, S835, K860, K1086 of SEQ ID NO.3

7. The CRISPR family protein site-specifically mutated by an unnatural amino acid of claim 4, wherein the unnatural amino acid is selected from one of the following compounds:

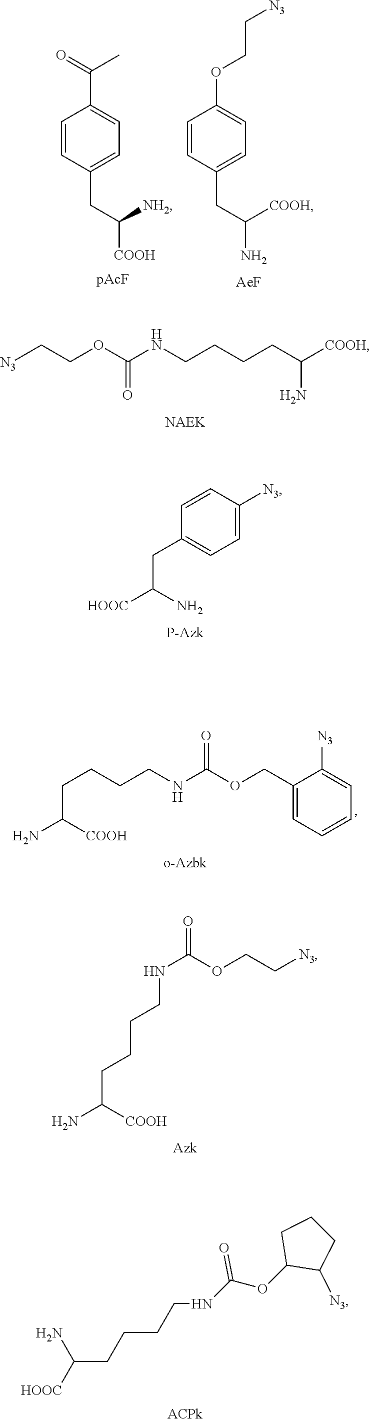

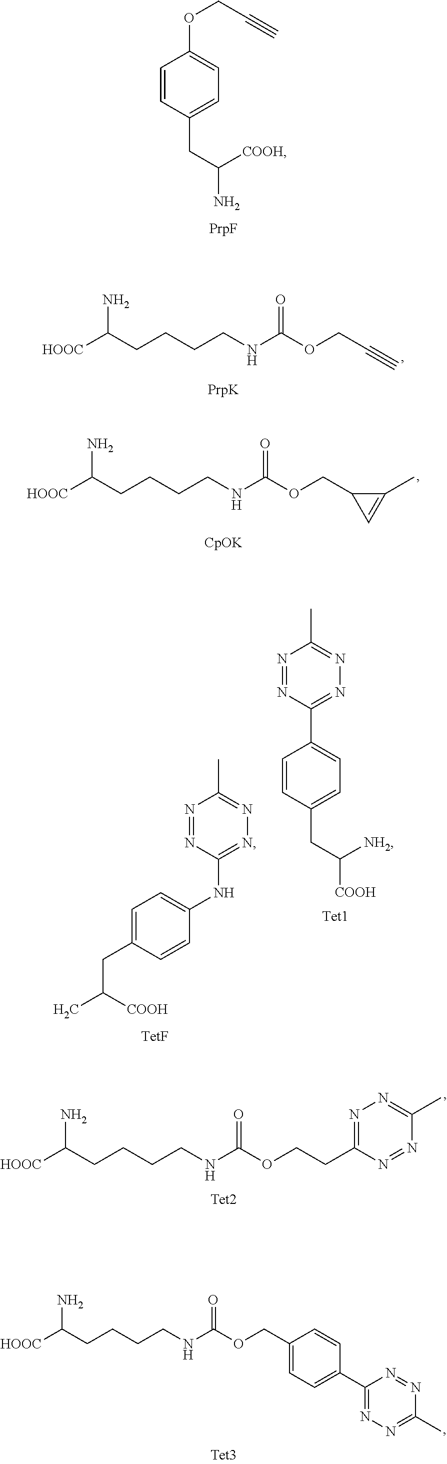

8. A method for preparing the CRISPR family protein site-specifically mutated by an unnatural amino acid of claim 4, comprising the steps of:
(1) site selection: based on structural information of the CRISPR family protein, selecting one or more specific amino acid sites in the amino acid sequence of the CRISPR family protein;
(2) genetic mutagenesis: mutating the codon encoding the amino acid at the site selected in step (1) to codon TAG by a genetic engineering method, to obtain a mutated CRISPR protein gene;
(3) construction of a mutant expression vector: ligating the mutated CRISPR protein gene obtained by genetic engineering in step (2) to an expression plasmid by means of molecular cloning, to obtain an expression vector plasmid comprising a mutant sequence; and
(4) protein expression: co-transfecting the expression vector plasmid obtained in (3) and a tool plasmid for unnatural amino acid insertion, into the same host cell, culturing the host cell successfully transfected in a culture solution supplemented with an unnatural amino acid, and inducing the expression of the mutated protein in the host cell.

9. The method for preparing the CRISPR family protein site-specifically mutated by an unnatural amino acid of claim 8, wherein the tool plasmid for unnatural amino acid insertion encodes tRNA and aminoacyl-tRNA synthetase, which specifically recognizes TAG codon and inserts, at the position corresponding to the codon, the unnatural amino acid.

10. A nucleic acid sequence encoding the CRISPR family protein site-specifically mutated by an unnatural amino acid of claim 4, wherein the codon corresponding to the unnatural amino acid in the base sequence of the nucleic acid is TAG, TAA or TGA.

11. A vector for preparing the CRISPR family protein site-specifically mutated by an unnatural amino acid of claim 4, comprising a corresponding nucleic acid sequence encoding the CRISPR family protein site-specifically mutated by an unnatural amino acid of claim 4.

12. A site-specific nucleic acid conjugate of a CRISPR family protein, wherein the CRISPR family protein is the CRISPR family protein site-specifically mutated by an unnatural amino acid of claim 4.

13. The site-specific nucleic acid conjugate of a CRISPR family protein of claim 12, wherein the nucleic acid is an oligo DNA, a donor DNA, a crRNA, and a related modified nucleic acid thereof.

14. Use of the CRISPR family protein site-specifically mutated by an unnatural amino acid of claim 4 or the site-specific nucleic acid conjugate thereof in improving gene editing efficiency and/or reducing off-target effect.

15. The method for site-specific conjugation of claim 1, wherein the related inactivated counterpart is dCas9 protein, dCas12a protein, or ddCas12a protein.

16. The CRISPR family protein site-specifically mutated by an unnatural amino acid of claim 4, wherein the related inactivated counterpart is dCas9 protein, dCas12a protein, or ddCas12a protein.

17. The CRISPR family protein site-specifically mutated by an unnatural amino acid of claim 7, wherein the unnatural amino acid is pAcF, AeF, PrpF, NAEK or TetF.

18. The method for preparing the CRISPR family protein site-specifically mutated by an unnatural amino acid of claim 8, wherein the CRISPR family protein is SpCas9 or AsCas12a.

19. The method for preparing the CRISPR family protein site-specifically mutated by an unnatural amino acid of claim 8, wherein the unnatural amino acid is pAcF, AeF, NAEK or another unnatural amino acid.

20. The nucleic acid sequence of claim 10, wherein the codon corresponding to the unnatural amino acid in the base sequence of the nucleic acid is TAG.
